SEQUENCE LISTING (2 sequences)

SEQ ID NO: 1 - protein, 47 aa, Artificial Sequence
  Synthetic construct
LSTAVKELVENSLDAGATNIDLKLKDYGVDLIEVSDNGCGVEEENFE

SEQ ID NO: 2 - protein, 50 aa, Artificial Sequence
  Synthetic construct
LRQVLSNLLDNAIKYTPEGGEITVSLERDGDHLEITVEDNGPGIPEEDLE

(19) United States
(12) Patent Application Publication
(10) Pub. No.: US 2008/0131427 A1
(43) Pub. Date: Jun. 5, 2008

(54) Whole Genome Evolution Technology Applied To Improve Protein And Antibody Yields By Cells

(75) Inventors: J. Bradford Kline, Norristown, PA (US); Qimin Chao, Havertown, PA (US); Philip M. Sass, Audubon, PA (US); Luigi Grasso, Bala Cynwyd, PA (US); Nicholas C. Nicolaides, Boothwyn, PA (US)

Correspondence Address: WOODCOCK WASHBURN LLP, CIRA CENTRE, 12TH FLOOR, 2929 ARCH STREET, PHILADELPHIA, PA, 19104-2891, US

(73) Assignee: Morphotek, Inc., Exton, PA (US)

(21) Appl. No.: 11/735,707

(22) Filed: Apr. 16, 2007

Related U.S. Application Data
(60) Provisional application No. 60/792,937, filed on Apr. 17, 2006.

(51) Int. Cl. A61K 39/395; C12N 5/06

(52) U.S. Cl. 424/133.1; 435/455; 435/325; 435/326

(57) ABSTRACT

Whole Genome Evolution Technology can be considered a broad tool for supporting the needs for scaleable manufacturing of therapeutic antibodies. Its random nature and in vivo mode of action separate this process from other complementary technologies, thus providing alternative solutions to improve a host cell's manufacturing performance. The speed with which a pre-existing production strain can be optimized makes this process suitable for satisfying the current need for rapid cell line optimization to produce faster growing cells exhibiting high titers of antibody at the preclinical, clinical or commercialization stage.

### CROSS-REFERENCE TO RELATED APPLICATIONS

This application claims benefit under 35 U.S.C. § 119(e) of U.S. Application Ser. No. 60/792,937, filed Apr. 17, 2006, which is incorporated herein by reference in its entirety.

### FIELD OF THE INVENTION

This invention relates to the generation of cells having improved growth properties (e.g., faster growth rates, enhanced biomolecule production at high cell density, and/or enhanced cell growth or viability at high cell density) relative to their parental cells. The invention further relates to screening of cells for improved growth properties for use in scaleable manufacturing and discovery of pathways involved in enhanced proliferation as it relates to cell-based manufacturing.

### BACKGROUND OF THE INVENTION

Therapeutic proteins and monoclonal antibodies (mAbs) have become one of the most successful classes of pharmaceutical agents over the past decade due to their ability to specifically replace or block a specific target associated with disease (1). With the successful market performance of commercial mAbs such as Remicade®, Rituxan®, Herceptin®, Humira®, and Avastin®, monoclonal antibodies have emerged as one of the most important drug classes and now represent about half of all new drug launches. Of all the mAbs launched to date, 40% are blockbuster drugs or have blockbuster revenue potential. Global sales of therapeutic mAbs have exceeded $10 billion in 2004 and are projected to be in excess of $30 billion by 2008 (Needham & Company, 2005). In the next five years, the mAb market will likely continue to be the fastest growing and most lucrative sector of the biotech and pharmaceutical industry, driven by technological evolution from chimeric (part human and part mouse) to humanized (CDR-grafted), to fully human antibodies. Compared to the traditional small molecule approach, mAbs offer greater specificity, less toxicity and a more rapid development path to the clinic. Unlike small molecules, which exert their function upon binding to a target, mAbs can have a greater therapeutic impact by utilizing the body's immune system to elicit target-specific cytotoxic activity.

Despite the huge success of mAbs, their efficient development remains quite challenging. Companies developing therapeutic mAbs must implement proper systems at an early stage of development for their successful GMP manufacturing, purification and finishing processes which ultimately impacts time-to-market, efficacy, safety and cost-of-goods. The production of therapeutic mAbs remains challenging due to the high manufacturing costs associated with annual yields required to support high doses for therapy and the high capital cost associated with adding production capacity. Manufacturing systems that can generate large quantities of product in a timely fashion are required for shorter development timelines and lower overall cost-of-goods that can be achieved by maintaining an abundance of fermentation capacity within the marketplace.

Companies specializing in mAb manufacturing have developed several cellular-based systems that can produce high mAb yields. These systems include bacterial, yeast, plant and mammalian cells (2). Microbial based platforms have resulted in some of the highest overall titer yields; however, the amount of active protein has typically represented only a fraction of the total protein generated because of improper folding or processing. In addition, microbial systems have not been successfully adapted to efficiently produce more complex proteins such as multichain macromolecules, including antibodies, which require additional processing such as post-translational modifications. In light of these microbial-based manufacturing limitations, mammalian cells remain one of the most reliable and widely used systems for large-scale, good manufacturing practice (GMP) manufacturing of therapeutic antibodies and a subset of non-antibody proteins.

There are currently 300 antibody development programs in progress that will require significant manufacturing capacity for clinical materials and ultimately commercial supply (3). The majority of these programs use recombinant mammalian cell lines such as Chinese Hamster Ovary (CHO) and mouse myeloma (NS0, SP2) cell lines under standard current GMP mammalian cell culture fermentation procedures using fed batch or perfusion bioreactors for manufacturing (4). Burden on cell-based manufacturing capacity continues to grow as product demands increase and each of these mAb programs progress through larger clinical trials and ultimately to the market. In light of this risk, alternative procedures are required to ensure that sufficient manufacturing capacity will continue to exist within the market and that the overall cost-of-goods remains similar or lower than current costs in today's market.

A variety of approaches are being pursued to support the future needs for large-scale manufacturing of antibodies from mammalian cells including the improvement of overall production and purification yields as well as the use of alternative manufacturing sources such as transgenic animal-based strategies (5, 6). While alternative host systems are being validated for cost-effective scaleability and regulatory compliance, the state-of-the-art remains mammalian cell culture. The ability to improve upon yields of antibody production in mammalian cells can be achieved by: 1) improving bioreactor performance via culturing conditions and/or media optimization; 2) improved vector expression by incorporating highly active promoters or increasing vector copy number by amplification; and/or 3) cell host optimization by enhancing endogenous pathways within the host cell line that provide for better titer yields and improved cell growth in large scale bioreactors. Any of these improvements or combinations thereof can result in processes that will shorten the number of manufacturing runs required to produce annual product needs, thereby relieving overall manufacturing constraints within the marketplace.

Cell host optimization can be achieved by manipulating endogenous pathways, including a) mRNA transcription and maturation, b) protein synthesis and post-translation modifications, c) protein secretion and cellular sub-localization, d) protein trafficking between cytosol and organelles, and e) cell cycle and survival regulation. Subtle structural changes in proteins involved in the regulation of one or more of these processes, as illustrated in FIG. 1, may directly or indirectly impact the overall performance of a production cell line. A randomized, genome-wide mutagenic approach that can be screened for functional cellular phenotypes offers an approach to enhancing complex cellular processes regulating growth rate, survival at very high cell density, protein synthesis and secretion rates. Unfortunately in most cell mutagenesis schemes, the use of mutagens results in genome-wide chromosomal instability yielding unstable cell lines that are not suitable for GMP manufacturing.

Previous studies have shown that inhibition of a post-replicative DNA repair mechanism, called mismatch repair, can lead to genetic diversity within stable cells due to increases in point mutations incorporated by DNA polymerase during DNA replication (7,8). DNA replication is a complex process that all cells undergo during proliferation in order for parental cells to pass on genetic information to sibling cells. As cells replicate their DNA, mutations occur within the newly synthesized template through a variety of mechanisms, including polymerase infidelity. A series of post-replicative DNA repair processes, such as the mismatch repair (MMR) system (11-13), have evolved in nature and are ubiquitously present in prokaryotic and eukaryotic cells in order for organisms to retain their genotypic identity. MMR prevents the accumulation of “naturally occurring” transition and transversion mutations via a secondary proof-reading process that corrects discordant genetic information that exists between parental and sibling DNA templates.

The process of inhibiting mismatch repair to generate genetic diversity within a cell population is referred to herein as “whole genome evolution technology.” Whole genome evolution technology is based on the reversible inhibition of MMR and is mediated by the activity of dominant negative protein inhibitors or chemical inhibitors that can block MMR within cells yielding, for example, novel therapeutic antibodies or proteins. Suppressed MMR results in the inheritance of point mutations in the genome of sibling cells due to mutations that occur during DNA replication (8,11,12). The suppression of MMR in cells allows naturally occurring mutations to be inherited at higher frequencies (up to 1000-fold enhancement) than typically observed in MMR-proficient cells. The genetically diverse population of sibling cells that are derived from whole genome evolution technology results in a library of cells that can be screened via automated functional high-throughput screening (HTS) to identify subclones with novel characteristics.

Whole genome evolution technology harnesses the power of evolution for the development of cells having desirable phenotypes. A key distinction that separates this technology from other evolution-based technologies is the random in vivo nature of the process. The ability to utilize the many genes and pathways that all cells innately possess permits the generation of unexpected mutants that are identified by functional cell screens, leading to sibling cells and gene products with desirable phenotypes. This technology is time and cost efficient because it can be applied in vivo to pre-existing production strains to enhance whole genome evolution thereby not requiring any in vitro manipulation. Whole genome evolution technology has been successfully applied to several mammalian cell lines producing recombinant therapeutic antibodies to derive evolved sibling cells with enhanced titer production, for example, in CHO, NSO, and hybridoma cells (8-10). Whole genome evolution technology also has been applied to mammalian cell lines producing recombinant therapeutic antibodies to derive evolved sibling cells that produce proteins or antibodies with improved biological properties (15-17).

Provided herein are, inter alia, processes that improve cell host performance to enhance productivity for antibody production by mammalian cell lines. Mismatch repair is inhibited in production cell lines to improve cellular processes affecting growth properties that can lead to improved antibody manufacturing yields.

### SUMMARY OF THE INVENTION

Provided herein are processes that improve cell host performance, for example, to enhance productivity by cell lines. The methods of the invention include methods for generating cells having at least one improved growth property relative to a parental cell population comprising: (a) inhibiting mismatch repair of a parental cell of a parental cell population; (b) incubating or expanding the MMR-inhibited parental cell to allow for mutagenesis, thereby generating hypermutated daughter cells; (c) detecting hypermutated daughter cells having the improved growth property; and (d) restoring genetic stability of hypermutated daughter cells having the improved growth property.

In some embodiments, the step of inhibiting mismatch repair of involves exposing the parental cell to a chemical inhibitor of mismatch repair or to a protein inhibitor of mismatch repair.

In some aspects of the invention, the step of incubating or expanding the MMR-inhibited parental cell to allow for mutagenesis, thereby generating hypermutated daughter cells, preferably comprises passaging the MMR-inhibited cells for at least 20 passages, more preferably at least 30 passages.

The step of restoring genetic stability in the methods of the invention preferably comprises withdrawing a chemical inhibitor of mismatch repair from the hypermutated daughter cell or inactivating a protein inhibitor of mismatch repair. Restoration of genetic stability may occur before, after, or simultaneously with the step of detecting hypermutated daughter cells having the improved growth property.

In the methods of the invention, the step of detecting hypermutated daughter cells having the improved growth property preferably comprises a high throughput screen (HTS) for hypermutated daughter cells having the improved growth property.

In preferred embodiments, the improved growth property is faster growth rate, enhanced cell growth or viability at high cell density, and/or enhanced production of a biomolecule at high cell density. In some embodiments, detection of hypermutated daughter cells having a faster growth rate comprises comparing the size of a cell population generated by a hypermutated daughter cell to the size of a cell population generated by a parental cell following an equivalent length of time in culture under like culture conditions, wherein a larger daughter cell population is indicative of the faster growth rate. In some embodiments, detection of hypermutated daughter cells having a faster growth rate comprises identifying the daughter cells having a growth ratio greater than a parental cell growth ratio. In some embodiments, detection of enhanced cell viability at high cell density comprises comparing the size of a viable cell population at high density generated by a hypermutated daughter cell to the size of a viable cell population at high density generated by a parental cell following an equivalent length of time in culture under like culture conditions, wherein a larger viable daughter cell population is indicative of enhanced cell viability at high cell density. Sizes of cell populations (e.g., parental cell populations and/or daughter cell populations) are preferably determined using an optical imaging system. In some embodiments, detection of hypermutated daughter cells exhibiting enhanced production of a biomolecule comprises determining a higher yield of the biomolecule by the daughter cells than by the parental cells under like culture conditions. In some embodiments, the sizes of the parental and daughter cell populations will be equivalent when determining the yield of biomolecule produced by each respective population.

Parental cells for use in the methods of the invention preferably are antibody-producing cells. In some embodiments, the parental cells are hybridoma cells. Parental cells may be eukaryotic or prokaryotic cells. Parental cells for use in the invention preferably are mammalian cells, more preferably human or rodent (e.g., hamster, mouse) cells, for example but not limited to NS0, SP2, or Chinese Hamster Ovary (CHO) cells. Parental cells for use in the methods of the invention may be bacterial cells, yeast cells, plant cells, or amphibian cells.

Also included within the scope of the invention are cells produced according to the methods of the invention and homogeneous and heterogeneous compositions of such cells.

Further provided in accordance with the invention are methods of manufacturing a biomolecule by culturing cells of the invention and isolating the biomolecule from the cells or culture medium of the cells. The biomolecule may comprise a chemical agent and/or a biological agent, such as but not limited to a protein, for example, an antibody. Biomolecules produced according to the methods of the invention and pharmaceutical compositions thereof are likewise included within the scope of the invention.

Methods of identifying genes responsible for an improved growth property comprising comparing the genome of the cells produced according to the methods of the invention to the genome of a parent cell to identify mutations, wherein a gene responsible for the improved growth property comprises at least one such mutation, also are included within the scope of the invention.

These and other aspects of the invention are provided by one or more of the embodiments described below.

### BRIEF DESCRIPTION OF THE DRAWINGS

FIG. 1 illustrates the pathways of antibody production in mammalian cells. Many pathways exist within mammalian cells that regulate protein production and cell growth during fermentation. Alteration of one or more of these pathways can be achieved through genetic manipulations to improve the production and growth performance of host cell lines. These pathways include: (1) gene transcription; (2) mRNA stability and translocation; (3) protein synthesis; (4) protein post-translation modification and trafficking; (5) folding within the endoplasmic reticulum (ER); (6) protein maturation and glycosylation within the golgi; (7) secretory pathways; and (8) cell membrane expression. (GlcNAc=N-acetylglucosamine; P,=phosphorylated residue).

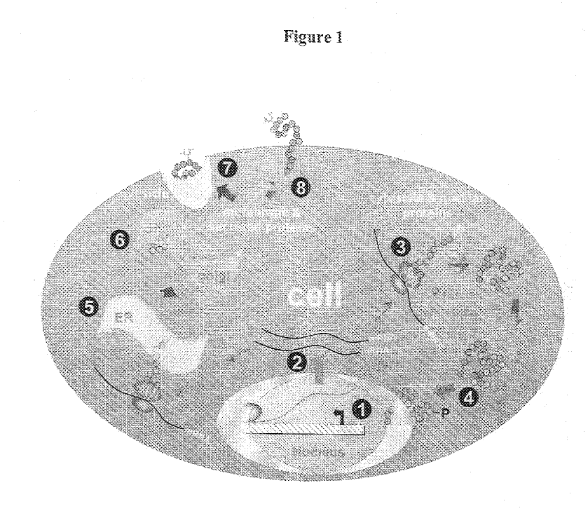

FIG. 2 illustrates production effects of cells with enhanced growth rates and stability at high cell density. A 10-day fermentation run was simulated for cells with enhanced growth rates or ability to grow at high density for extended periods. The model assumes that i) as the cell density increases, the growth rate decreases; ii) accumulation of antibody depends solely on cell number and pcd; and iii) seeding at day 0 is 500,000/mL. The doubling time of the parental line is 32 hours, maximal density of 1.7×10⁶/mL is reached at day 5, and its specific productivity is 25 pg/cell/day (pcd). Under these conditions, a parental cell line would yield approximately 0.28 g/L upon completion of a fermentation run. As shown here, a cell line derived from the parental line that reached a higher cell density (HD) of 3.4×10⁶/mL at day 7 would have a higher antibody titer, in this graph represented by the sphere's size and number adjacent expressing gram per liter. Another line with a faster growth rate (FGR), or faster doubling time, of 24 hour versus 32 hours would produce an even higher yield than that achieved by cells capable of growing at higher density. A cell line exhibiting a combined improvement in high density growth and growth rate (HD and FGR, respectively) would perform best in this model, reaching 3.32 g/L.

FIG. 3 shows the whole genome evolution technology process flow chart.

FIG. 4 demonstrates cell counting via microscopic image analysis. CHO-MAb cells were seeded in 96 well U-bottom plates at densities of 100 to 10,000 cells per well to determine linearity of imaging program. Wells were imaged at 20× using the Meta Imaging System. Colony size was determined by integrating the pixel area of the well covered by cells using the Metamorph software package (Ver.6.3r0). Data points are average of 12 wells±standard deviation.

FIG. 5 compares colony size of CHO-MAb parental or whole genome evolution technology-derived clones. Parental or whole genome evolution technology CHO-MAb cells are subcloned into 96 well bar-coded plates and grown for 12 days in a CO₂ incubator at 37° C. Representative CHO-MAb colonies are imaged on day 14 and then re-imaged on day 17 using Metamorph Imaging software that calculates pixel area of colonies. The growth ratio is determined by dividing the area of colonies at day 17 to the area of its representative clone at day 14 by 3 days (Growth ratio=day 17 area/day 14 area÷3 days). Parental cells for the CHO-MAb line have a growth ratio of ˜0.5. Cells exhibiting growth ratios of >0.8 are expanded and analyzed in standardized growth assays. The inherent slower growth rate of parental cells typically result in smaller colonies at day 14 in contrast to clones that have evolved to have a faster growth as expected.

FIG. 6 illustrates representative growth data confirming faster growing subclones. Faster growth rate and parental CHO-MAb cells were grown in a 7-day shake flask assays and analyzed for growth rates by cell counting at days 1, 4 and 7. Shown are growth rates as a function of doubling time in hours for a subset of Faster growth rate (WGET-1 and WGET-2) and parental cell-derived (Parental-1 and Parental-2) CHO-MAb subclones.

### DETAILED DESCRIPTION OF ILLUSTRATIVE EMBODIMENTS

The reference works, patents, patent applications, and scientific literature, including accession numbers to GenBank database sequences that are referred to herein establish the knowledge of those with skill in the art and are hereby incorporated by reference in their entirety to the same extent as if each was specifically and individually indicated to be incorporated by reference. Any conflict between any reference cited herein and the specific teachings of this specification shall be resolved in favor of the latter. Likewise, any conflict between an art-understood definition of a word or phrase and a definition of the word or phrase as specifically taught in this specification shall be resolved in favor of the latter.

Each range recited herein includes all combinations and sub-combinations of ranges, as well as specific numerals contained therein.

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology will be used.

As used in this specification and the appended claims, the singular forms “a”, “an” and “the” include plural referents unless the content clearly dictates otherwise. Thus, for example, reference to “a cell” includes a combination of two or more cells, and the like.

The term “about” as used herein when referring to a measurable value such as an amount, a temporal duration, and the like, is meant to encompass variations of ±20% or 110%, more preferably ±5%, even more preferably 1%, and still more preferably ±0.1% from the specified value, as such variations are appropriate to perform the disclosed methods.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

“Recombinant” when used with reference, e.g., to a cell, nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, underexpressed or not expressed at all.

The phrase “nucleic acid” or “polynucleotide sequence” refers to a single or double-stranded polymer of deoxyribonucleotide or ribonucleotide bases read from the 5′ to the 3′ end. Nucleic acids can also include modified nucleotides that permit correct readthrough by a polymerase and do not alter expression of a polypeptide encoded by that nucleic acid, including, for example, conservatively modified variants.

“Polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymers. Polypeptides include conservatively modified variants. One of skill will recognize that substitutions, deletions or additions to a nucleic acid, peptide, polypeptide, or protein sequence which alter, add or delete a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention. The following eight groups each contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (33). The term “conservative substitution” also includes the use of a substituted amino acid in place of an unsubstituted parent amino acid provided that such a polypeptide also displays the requisite binding activity.

“Amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. “Amino acid analog” refers to compounds that have the same basic chemical structure as a naturally occurring amino acid, i.e., an α carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones but retain the same basic chemical structure as a naturally occurring amino acid. “Amino acid mimetic” refers to a chemical compound having a structure that is different from the general chemical structure of an amino acid but that functions in a manner similar to a naturally occurring amino acid.

Amino acids can be referred to herein by either their commonly known three letter symbols or by the one-letter symbols recommended by the IUPAC-IUB Biochemical Nomenclature Commission (see Table 1 below). Nucleotides, likewise, can be referred to by their commonly accepted single-letter codes.

TABLE 1
SYMBOL

| 1-Letter | 3-Letter | AMINO ACID |
|---|---|---|
| Y | Tyr | L-tyrosine |
| G | Gly | L-glycine |
| F | Phe | L-phenylalanine |
| M | Met | L-methionine |
| A | Ala | L-alanine |
| S | Ser | L-serine |
| I | Ile | L-isoleucine |
| L | Leu | L-leucine |
| T | Thr | L-threonine |
| V | Val | L-valine |
| P | Pro | L-proline |
| K | Lys | L-lysine |
| H | His | L-histidine |
| Q | Gln | L-glutamine |
| E | Glu | L-glutamic acid |
| W | Trp | L-tryptophan |
| R | Arg | L-arginine |
| D | Asp | L-aspartic acid |
| N | Asn | L-asparagine |
| C | Cys | L-cysteine |

It should be noted that all amino acid sequences are represented herein by formulae whose left to right orientation is in the conventional direction of amino-terminus to carboxy-terminus.

As used herein, the term “in vitro” or “ex vivo” refers to an artificial environment and to processes or reactions that occur within an artificial environment, for example, but not limited to, test tubes and cell cultures. The term “in vivo” refers to a natural environment (e.g., an animal or a cell) and to processes or reactions that occur within a natural environment.

“Pharmaceutically acceptable,” “physiologically tolerable” and grammatical variations thereof, as they refer to compositions, carriers, diluents and reagents, are used interchangeably and represent that the materials are capable of administration to or upon a human without the production of undesirable physiological effects to a degree that would prohibit administration of the composition.

The term “pharmaceutically acceptable carrier” refers to reagents, excipients, cells, compounds, materials, compositions, and/or dosage forms which are, within the scope of sound medical judgment, suitable for use in contact with the tissues of human beings and animals without excessive toxicity, irritation, allergic response, or other complication commensurate with a reasonable benefit/risk ratio. As described in greater detail herein, pharmaceutically acceptable carriers suitable for use in the present invention include gases, liquids, and semi-solid and solid materials.

“Immunoglobulin” or “antibody” is used broadly to refer to both antibody molecules and a variety of antibody-derived molecules and includes any member of a group of glycoproteins occurring in higher mammals that are major components of the immune system. The term “antibody” is used in the broadest sense and specifically covers monoclonal antibodies, antibody compositions with polyepitopic specificity, bispecific antibodies, diabodies, and single-chain molecules, as well as antibody fragments (e.g., Fab, F(ab′)2, and Fv), so long as they exhibit the desired biological activity (e.g. specific binding to target antigen). An immunoglobulin molecule includes antigen binding domains, which each include the light chains and the end-terminal portion of the heavy chain, and the Fc region, which is necessary for a variety of functions, such as complement fixation. There are five classes of immunoglobulins wherein the primary structure of the heavy chain, in the Fc region, determines the immunoglobulin class. Specifically, the alpha, delta, epsilon, gamma, and mu chains correspond to IgA, IgD, IgE, IgG and IgM, respectively. As used herein “immunoglobulin” or “antibody” includes all subclasses of alpha, delta, epsilon, gamma, and mu and also refers to any natural (e.g., IgA and IgM) or synthetic multimers of the four-chain immunoglobulin structure. Antibodies non-covalently, specifically, and reversibly bind an antigen. The term “monoclonal antibody” as used herein refers to an antibody obtained from a population of substantially homogeneous antibodies, i.e., the individual antibodies comprising the population are identical except for possible naturally occurring mutations that can be present in minor amounts. For example, monoclonal antibodies may be produced by a single clone of antibody-producing cells. Unlike polyclonal antibodies, monoclonal antibodies are monospecific (e.g., specific for a single epitope of a single antigen). The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention can be made by the hybridoma method first described by Kohler et al. (18) or can be made by recombinant DNA methods. The “monoclonal antibodies” can also be isolated from phage antibody libraries using the techniques described in Marks et al. (19), for example.

Antibody-derived molecules comprise portions of intact antibodies that retain antigen-binding specificity, and comprise, for example, at least one variable region (either a heavy chain or light chain variable region). Antibody-derived molecules, for example, include molecules such as Fab fragments, Fab′ fragments, F(ab′)2 fragments, Fd fragments, F(v) fragments, Fabc fragments, Fd fragments, Fabc fragments, Sc antibodies (single chain antibodies), diabodies, individual antibody light chains, individual antibody heavy chains, chimeric fusions between antibody chains and other molecules, heavy chain monomers or dimers, light chain monomers or dimers, dimers consisting of one heavy and one light chain, and the like. All classes of immunoglobulins (e.g. IgA, IgD, IgE, IgG and IgM) and subclasses thereof are included.

Antibodies can be labeled/conjugated to toxic or non-toxic moieties. Toxic moieties include, for example, bacterial toxins, viral toxins, radioisotopes, and the like. Antibodies can be labeled for use in biological assays (e.g., radioisotope labels, fluorescent labels) to aid in detection of the antibody. Antibodies can also be labeled/conjugated for diagnostic or therapeutic purposes, e.g., with radioactive isotopes that deliver radiation directly to a desired site for applications such as radioimmunotherapy (20), imaging techniques and radioimmunoguided surgery or labels that allow for in vivo imaging or detection of specific antibody/antigen complexes. Antibodies may also be conjugated with toxins to provide an immunotoxin (21).

With respect to antibodies, the term, “immunologically specific” refers to antibodies that bind to one or more epitopes of a target molecule, but which do not substantially recognize and bind other molecules in a sample containing a mixed population of antigenic biological molecules.

“Chimeric” or “chimerized” antibodies (immunoglobulins) refer to antibodies in which a portion of the heavy and/or light chain is identical or homologous to corresponding sequences in antibodies derived from a particular species or belonging to a particular antibody class or subclass, while the remainder of the chain(s) is identical or homologous to corresponding sequences in antibodies derived from another species or belonging to another antibody class or subclass, as well as fragments of such antibodies, so long as they exhibit the desired biological activity (22).

“Humanized” forms of non-human (e.g. murine) antibodies are chimeric immunoglobulins, immunoglobulin chains or fragments thereof (such as Fv, Fab, Fab′, F(ab′)₂ or other antigen-binding subsequences of antibodies) which contain minimal sequence derived from non-human immunoglobulin. For the most part, humanized antibodies are human immunoglobulins (recipient antibody) in which residues from a complementary-determining region (CDR) of the recipient are replaced by residues from a CDR of a non-human species (donor antibody) such as mouse, rat or rabbit having the desired specificity, affinity, and capacity. In some instances, Fv framework region (FR) residues of the human immunoglobulin are replaced by corresponding non-human residues. Furthermore, humanized antibodies can comprise residues which are found neither in the recipient antibody nor in the imported CDR or framework sequences. These modifications are made to further refine and optimize antibody performance. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin sequence. The humanized antibody optimally also will comprise at least a portion of an immunoglobulin constant region (Fc), typically that of a human immunoglobulin. For further details, see Jones et al. (23); Reichmann et al., (24); Presta (25).

“Fully human” refers to an immunoglobulin, such as an antibody, where the whole molecule is of human origin or consists of an amino acid sequence identical to a human form of the antibody.

“Epitope” refers to an immunological determinant of an antigen that serves as an antibody-binding site. As used herein, the term “conformational epitope” refers to a discontinuous epitope formed by a spatial relationship between amino acids of an antigen other than an unbroken series of amino acids.

“Hybridoma” refers to the product of a cell-fusion between a cultured neoplastic lymphocyte and a primed B- or T-lymphocyte which expresses the specific immune potential of the parent cell.

As used herein, “high titer” refers to a titer of at least about 1.5 fold higher than the parental cell line. In some embodiments, the titer is at least about 1.5-3 fold higher, 3-5 fold higher, 5-7 fold higher, 7-9 fold higher, or 9-10 fold higher than the parental cell line.

As used herein, “high affinity” refers to a high antibody binding affinity, that may be calculated according to standard methods by the formula Kₐ=8/3 (Iₜ−Tₜ) where “Iₜ” is the total molar concentration of inhibitor uptake at 50% tracer and “Tₜ” is the total molar concentration of tracer (26). Binding affinity may also be calculated using the formula B/T=n·N_{Ab}·W¹⁰⁸[(V−Vₘ)K+Q·W] (27). As used herein, “high affinity” is less than about 1×10⁷ M⁻¹ In some embodiments, the antibodies have an affinity of less than about 1×10⁸ M⁻¹. In other embodiments, the antibodies have an affinity of less than about 1×10⁹ M⁻¹. In other embodiments, the antibodies have an affinity of less than about 1×10¹⁰ M⁻¹. In other embodiments, the antibodies have an affinity of less than about 1×10¹¹ M⁻¹. In other embodiments, the antibodies have an affinity of less than about 1×10¹² M⁻¹. In other embodiments, the antibodies have an affinity of less than about 1×10¹³ M⁻¹. In other embodiments, the antibodies have an affinity of less than about 1×10¹⁴ M¹.

As used herein, the term “faster growth rate” refers to a state in which a given population of cells, e.g., a daughter population such as a hypermutated daughter cell population, exhibits a growth ratio greater than a reference cell population, e.g., a parental cell population such as a parental cell population in which mismatch repair has not been inhibited, or in which a given population demonstrates a faster doubling time than a reference cell population under like culture conditions (e.g., temperature, culture medium, length of culture period, humidity, CO₂, O₂, etc.). “Growth ratio” generally can be calculated by dividing the area of a cell colony determined at a first timepoint into the area of the cell colony at a second later timepoint and dividing the resulting number by the difference in time between the two respective timepoints. For example, growth ratio may be determined by dividing the area of colonies at day 17 to the area of its representative clone at day 14 by 3 days (Growth ratio=day 17 area/day 14 area÷3 days).

As used herein, the term “enhanced high density production” or “enhanced production of a biomolecule at high cell density” refers to a state in which a given cell population, e.g. a daughter population such as a hypermutated daughter cell population, exhibits a greater production of a biomolecule (e.g., antibody) than a reference cell population, e.g. a parental cell population such as a parental cell population in which mismatch repair has not been inhibited, under like culture conditions (e.g. temperature, culture medium, length of culture period, humidity, CO₂, O₂, etc.).

As used herein, “enhanced cell growth at high cell density” refers to a state in which a given cell population, e.g., a daughter cell population such as a hypermutated daughter cell population, exhibits increased levels of growth, for example, as measured by cell number, relative to a reference population, e.g., a parental cell population such as a parental cell population in which mismatch repair has not been inhibited, under like culture conditions (e.g., temperature, culture medium, length of culture period, humidity, CO₂, O₂, etc.).

As used herein, “enhanced cell viability at high cell density” refers to a state in which a given cell population, e.g., a daughter cell population such as a hypermutated daughter cell population, exhibits increased numbers of viable cells relative to a reference population, e.g., a parental cell population such as a parental cell population in which mismatch repair has not been inhibited, under like culture conditions (e.g., temperature, culture medium, length of culture period, humidity, CO₂, O₂, etc.).

As used herein, “high cell density” refers to a cell concentration of greater than about 1.0×10⁶ cells/mL. “Low cell density” refers to a cell concentration less than about 1.0×10⁶ cells/mL.

As used herein, “cured” refers to a state of cells wherein the protein inhibitor of mismatch repair has been eliminated from the cell or wherein the expression of the protein inhibitor of mismatch repair has been turned off or knocked out, leading to a stabilized genome, producing stable biological products such as immunoglobulins. Similarly, “inactivation” of an inhibitor of mismatch repair refers to elimination or removal of the inhibitor from the cell or discontinued expression of a protein inhibitor of mismatch repair (e.g. turn off or knock out expression of the inhibitor), leading to a stabilized genome, producing stable biomolecules such as immunoglobulins.

As used herein, “screening” refers to an assay to assess the genotype or phenotype of a cell or cell product including, but not limited to nucleic acid sequence, protein sequence, protein function (e.g. binding, enzymatic activity, blocking activity, cross-blocking activity, neutralization activity, and the like). The assays include ELISA-based assays, Biacore analysis, and the like.

As used herein, “isolated” refers to a nucleic acid or protein that has been separated and/or recovered from a component of its natural environment. Contaminant components of its natural environment are materials which would interfere with diagnostic or therapeutic uses for the polypeptide, and may include enzymes, hormones, and other proteinaceous or non-proteinaceous solutes. In some embodiments, the nucleic acid or protein is purified to greater than 95% by weight of protein. In other embodiments, the nucleic acid or protein is purified to greater than 99% by weight of protein. Determination of protein purity may be by any means known in the art such as the Lowry method, by SDS-PAGE under reducing or non-reducing conditions using a stain such as a Coomassie blue or silver stain. Purification of nucleic acid may be assessed by any known method, including, but not limited to spectroscopy, agarose or polyacrylamide separation with fluorescent or chemical staining such as methylene blue, for example.

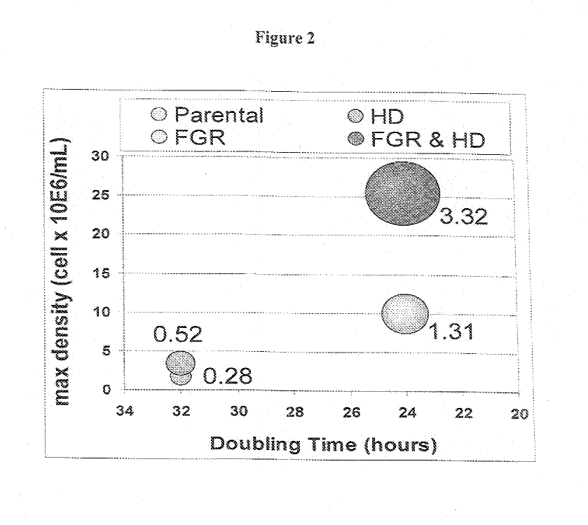

Provided herein are processes that improve cell host performance to enhance productivity for antibody production by mammalian cell lines. Mismatch repair is inhibited in production cell lines to improve cellular processes affecting growth properties that can lead to improved manufacturing yields. For example, cells can be genetically enhanced for faster growth, growth and/or viability at high cell density, and/or the ability to maintain productivity at high density for longer fermentation runs. While not intending to be bound by any theory, it is believed that these enhancements occur via structural changes within growth factors or biochemical receptors sensing accumulation of metabolic byproducts that perturb growth and survival. FIG. 2 provides a model of the dramatic effects of enhanced growth by Faster Growth Rate (FGR) cells and/or cells that grow and produce at a High Density (HD) on overall productivity for scaleable manufacturing.

The methods provided herein to improve growth properties including growth rate, cell growth and viability at high cell density, and/or productivity at high cell density within production cell lines, preferably mammalian production cell lines, confer on cells the ability to produce higher yields of monoclonal antibodies (mAbs) in a timely and cost effective manner. Cells having enhanced cell growth parameters for improving overall production at scale are provided. The methods described herein improve growth rates of recombinant mammalian cells while having little impact on specific productivity.

Provided herein are methods for generating cells having improved growth characteristics, including at least one growth property of faster growth rate, enhanced cell growth and viability at high cell density, and enhanced biomolecule production at high cell density. In some preferred embodiments, the improved growth characteristics include faster growth rate and enhanced biomolecule production at high cell density; faster growth rate and enhanced cell growth and viability at high cell density; enhanced cell growth and viability at high cell density and enhanced biomolecule production at high cell density; or faster growth rate, enhanced cell growth and viability at high cell density, and enhanced biomolecule production at high cell density. In some embodiments, the cells further demonstrate at least one characteristic of production of high affinity antibodies and high titer antibody production. The methods for generating cells having improved growth characteristics comprise application of whole genome evolution technology as taught herein. An advantage of whole genome evolution technology is that it can be directly applied to a cell line, for example a cell line expressing antibodies (i.e., a production cell line), preferably a mammalian cell line, more preferably a mammalian cell line expressing antibodies (i.e., a mammalian production cell line or mammalian manufacturing cell line) for which there is a need to further optimize growth characteristics, titer yields for scaleable manufacturing, or both. The invention facilitates the generation of enhanced growth antibody-producing cell lines for manufacturing. Whole genome evolution technology may be applied to a cell line for producing sufficient antibody quantities for timely, scaleable GMP manufacturing. Whole genome evolution technology can be applied to optimize host growth parameters of cells of any species by virtue of the high degree of conservation and function of MMR in microbial, plant and mammalian cell-based systems.

The methods for generating genetically altered host cells having improved growth properties provide a valuable method for creating cell hosts for product development as well as allow for the generation of reagents useful for the discovery of downstream genes whose altered structure or expression levels when altered result in enhanced cell growth properties. The invention described herein is directed to the creation of genetically altered cell hosts with enhanced growth properties via the blockade of MMR that can in turn be used to screen and identify altered gene loci for directed alteration and generation of enhanced growth strains.

To enhance genetic evolution in mammalian cells, MMR is suppressed in the host cell. MMR is preferably suppressed by introducing a protein inhibitor of MMR (e.g., by introducing an expression vector encoding a MMR inhibitory protein) or by exposing (e.g., incubating cells in the presence of a chemical inhibitor of MMR) host cells to a chemical MMR inhibitor (8,9). Both methods have been shown to be effective in inhibiting MMR and resulting in genetically evolved sibling cells and have been used interchangeably (L. Grasso, personal observations).

Host cells (i.e., parental cells) may be eukaryotic or prokaryotic. Preferably, host cells are mammalian cells, more preferably human or rodent (e.g., mouse, hamster) cells. Mammalian cells suitable for use in certain embodiments of the method of the invention include, but are not limited to sp20 cells, NS0 cells, Chinese Hamster Ovary cells (CHO cells (28)), baby hamster kidney (BHK cells), human embryonic kidney line 293 (HeLa cells (29)), normal dog kidney cell line (e.g., MDCK, ATCC CCL 34), normal cat kidney cell line (CRFK cells), monkey kidney cells (CV1 ATCC CCL 70); African green monkey kidney cells (VERO-76, ATCC CRL-1587), COS (e.g., COS-7) cells, and non-tumorigenic mouse myoblast G8 cells (e.g., ATCC CRL 1246), fibroblast cell lines (e.g. human, murine or chicken embryo fibroblast cell lines), myeloma cell lines, mouse NIH/3T3 cells, LMTK³¹ cells, mouse sertoli cells (TM4, (30)); human cervical carcinoma cells (HELA, ATCC CCL 2); buffalo rat liver cells (BRL 3A, ATCC CRL 1442); human lung cells (W138, ATCC CCL 75); human liver cells (Hep G2, HB 8065); mouse mammary tumor cells (MMT 060562, ATCC CCL51), TR1 cells (31); MRC 5 cells; FS4 cells; and the human hepatoma line (Hep G2).

As an alternative to mammalian expression cells, other non-mammalian cells may be used in the methods of the invention. Such non-mammalian cells include, but are not limited to, eukaryotic and prokaryotic cells including bacterial cells, yeast cells, plant cells, amphibian cells, and insect cells (e.g., Spodoptera frugiperda cells and the like). Vectors and non-mammalian host cells are well known in the art and are continually being optimized and developed. Any host cell system capable of expressing antibodies may be used in the methods of the invention.

Host cells preferably are antibody-producing cells. For example, host cells may be hybridoma cells.

Cells may be rendered hypermutable by the introduction of a protein inhibitor of mismatch repair (MMR). For example, the protein inhibitor may be encoded by a polynucleotide comprising a dominant negative allele of a MMR gene. The nucleic acid encoding the protein inhibitor of MMR may be introduced into a cell, for example, an expression cell, a hybridoma cell (i.e., after the fusion of immunoglobulin-producing cells with the myeloma cells), or a myeloma cell (i.e., may be introduced into the cell prior to fusion).

The nucleic acid encoding the protein inhibitor of MMR may be genomic DNA, cDNA, RNA, or a chemically synthesized polynucleotide. The polynucleotide can be cloned into an expression vector containing a constitutively active promoter segment (such as, but not limited to, CMV, SV40, EF-1 Dor LTR sequences) or an inducible promoter sequence such as those from tetracycline, or ecdysone/glucocorticoid inducible vectors, wherein expression of the inhibitor can be regulated. The polynucleotide can be introduced into the cell by transfection.

Transfection is any process whereby a polynucleotide is introduced into a cell. The process of transfection can be carried out in vitro, e.g. using a suspension of one or more isolated cells in culture. The cell can be any immortalized cell used for creating hybridomas for the production of monoclonal antibodies, or the cell may be the hybridoma itself. The hybridomas may be heterohybridoma cells (e.g. human-mouse cell fusions) or homohybridoma cells (e.g., human-human hybridoma cells and mouse-mouse hybridoma cells).

In general, transfection will be carried out using a suspension of cells, or a single cell, but other methods can also be applied as long as a sufficient fraction of the treated cells or tissue incorporates the polynucleotide so as to allow transfected cells to be grown and utilized. The protein product of the polynucleotide may be transiently or stably expressed in the cell. Techniques for transfection are well known. Available techniques for introducing polynucleotides include but are not limited to electroporation, transduction, cell fusion, the use of calcium chloride, and packaging of the polynucleotide together with lipid for fusion with the cells of interest. Once a cell has been transfected with the nucleic acid encoding the protein inhibitor of mismatch repair, the cell can be grown and reproduced in culture. If the transfection is stable, such that the nucleic acid encoding the protein inhibitor of mismatch repair is expressed at a consistent level for many cell generations, then a cell line results.

The nucleic acid encoding the dominant negative protein inhibitor of mismatch repair may be derived from any known mismatch repair gene including, but not limited to PMS2, PMS2-134, PMS1, PMSR3, PMSR2, PMSR6, MLH1, GTBP, MSH3, MSH2, MLH3, or MSH1, and homologs of PMSR genes as described in U.S. Pat. Nos. 6,146,894 and 6,808,894 and U.S. Publ. Appl. Nos. 20040115695 and 20050048621, each of which is incorporated herein by reference; Nicolaides et al. (32) and Horii et al. (33). Any allele which produces such effect can be used in this invention. The dominant negative protein inhibitor of mismatch repair can be obtained from the cells of humans, animals, yeast, bacteria, or other organisms. A non-limiting example of a protein inhibitor of mismatch repair is PMS2-134 having the first 133 amino acids of PMS2. PMS2 and PMS2-134 as used herein include human PMS2 and PMS2-134 and species equivalents thereof (e.g., mouse; plant, such as Arabidopsis thaliana; etc.). The lack of the C-terminus in the PMS2 protein is believed to interfere with the binding of PMS2 with MLH1. Further delineation of amino acids in mutL homologs that inhibit mismatch repair reveals common amino acid sequences LSTAVKELVENSLDAGATNIDLKLKDYGVDLIEVSDNGCGVEEENFE (SEQ ID NO:1) and LRQVLSNLLDNAIKYTPEGGEITVSLERDGDHLEITVEDNGPGIPEEDLE (SEQ ID NO:2) or fragments thereof. Protein inhibitors of mismatch repair thus include polypeptides comprising amino acid sequences of SEQ ID NO:1 or 2 and fragments thereof.

Screening cells for defective mismatch repair activity can identify additional protein inhibitors of mismatch repair. Cells from animals or humans with cancer can be screened for defective mismatch repair. Cells from colon cancer patients may be particularly useful. Genomic DNA, cDNA, or mRNA from any cell encoding a protein inhibitor of mismatch repair can be analyzed for variations from the wild type sequence. Dominant negative alleles of a mismatch repair gene can also be created artificially, for example, by producing variants of the hPMS2-134 allele or other mismatch repair genes. Various techniques of site-directed mutagenesis can be used. The suitability of such alleles, whether natural or artificial, for use in generating hypermutable cells or animals can be evaluated by testing the mismatch repair activity caused by the allele in the presence of one or more wild-type alleles, to determine if it is a dominant negative allele.

Dominant negative protein inhibitors of mismatch repair increase the rate of spontaneous mutations by reducing the effectiveness of DNA repair and thereby render the cells or animals hypermutable. This means that the spontaneous mutation rate of such cells or animals is elevated compared to cells or animals without such alleles. The degree of elevation of the spontaneous mutation rate can be at least 2-fold, 5-fold, 10-fold, 20-fold, 50-fold, 100-fold, 200-fold, 500-fold, or 1000-fold that of the normal cell or animal. The hypermutable cells will accumulate new mutations in gene(s) to produce new output traits. The cells can be screened for desired characteristics and cell lines bearing these characteristics may be expanded. Furthermore, the cells may be cured of the mismatch repair defect, leading to genetically stable cells. In preferred embodiments, the protein inhibitor of mismatch repair is inactivated. For example, the protein inhibitor of mismatch repair may be inactivated before or after identification of a cell having the desired growth properties. Inactivation of the protein inhibitor of mismatch repair may be by any means known in the art, for example, removal of an inducer or removal of the protein inhibitor of mismatch repair from the cell (i.e., curing the cell of the protein inhibitor of mismatch repair). Inactivation of the inhibitor of mismatch repair stabilizes the genome of the hypermutated cell.

Another aspect of the invention is the use of cells lacking MMR (either due to defects in endogenous mismatch repair genes, or due to the introduction of dominant negative protein inhibitors of MMR) and chemical mutagens to cause an enhanced rate of mutation in a host's genome. The lack of MMR activity has been known to make cells more resistant to the toxic effects of DNA damaging agents. This invention comprises making proficient MMR cells mismatch repair defective via the expression of a dominant negative protein inhibitor of MMR and then enhancing the genomic hypermutability with the use of a DNA mutagen. Chemical mutagens are classifiable by chemical properties, e.g. alkylating agents, cross-linking agents, etc. The following chemical mutagens are useful, as are others not listed here, according to the invention and may be used to further enhance the rate of mutation in any of the embodiments of the method of the invention: N-ethyl-N-nitrosourea (ENU), N-methyl-N-nitrosourea (MNU), procarbazine hydrochloride, chlorambucil, cyclophosphamide, methyl methanesulfonate (MMS), ethyl methanesulfonate (EMS), diethyl sulfate, acrylamide monomer, triethylene melamin (TEM), melphalan, nitrogen mustard, vincristine, dimethylnitrosamine, N-methyl-N′-nitro-nitrosoguanidine (MNNG), 7,12 dimethylbenz (a) anthracene (DMBA), ethylene oxide, hexamethylphosphoramide, bisulfan. In a preferred aspect of the invention, a mutagenesis technique is employed that confers a mutation rate in the range of 1 mutation out of every 100 genes; 1 mutation per 1,000 genes. The use of such combination (MMR deficiency and chemical mutagens) will allow for the generation of a wide array of genome alterations (such as but not limited to expansions or deletions of DNA segments within the context of a gene's coding region, a gene's intronic regions, or 5′ or 3′ proximal and/or distal regions, point mutations, altered repetitive sequences) that are preferentially induced by each particular agent.

Mutations can be detected by analyzing for alterations in the genotype of the cells or animals, for example by examining the sequence of genomic DNA, cDNA, messenger RNA, or amino acids associated with the gene of interest. Mutations can also be detected by screening the phenotype of the gene. An altered phenotype can be detected by identifying alterations in electrophoretic mobility, spectroscopic properties, or other physical or structural characteristics of a protein encoded by a mutant gene. One can also screen for altered function of the protein in situ, in isolated form, or in model systems. One can screen for alteration of any property of the cell or animal associated with the function of the gene of interest, such as but not limited to measuring protein secretion, chemical-resistance, pathogen resistance, etc.

In some embodiments of the methods of generating cells having improved growth properties of the invention, the cells are exposed to a chemical inhibitor of mismatch repair. Chemical inhibitors of mismatch repair used in certain embodiments of the methods of the invention include, but are not limited to, at least one of an anthracene, an ATPase inhibitor, a nuclease inhibitor, an RNA interference molecule, a polymerase inhibitor and an antisense oligonucleotide that specifically hybridizes to a nucleotide encoding a mismatch repair protein (WO2004/046330). In preferred embodiments, the chemical inhibitor is an anthracene compound having the formula:

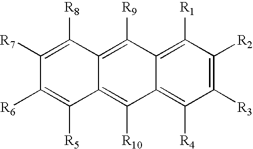

wherein R₁-R₁₀ are independently hydrogen, hydroxyl, amino, alkyl, substituted alkyl, alkenyl, substituted alkenyl, alkynyl, substituted alkynyl, O-alkyl, S-alkyl, N-alkyl, O-alkenyl, S-alkenyl, N-alkenyl, O-alkynyl, S-alkynyl, N-alkynyl, aryl, substituted aryl, aryloxy, substituted aryloxy, heteroaryl, substituted heteroaryl, aralkyloxy, arylalkyl, alkylaryl, alkylaryloxy, arylsulfonyl, alkylsulfonyl, alkoxycarbonyl, aryloxycarbonyl, guanidino, carboxy, an alcohol, an amino acid, sulfonate, alkyl sulfonate, CN, NO₂, an aldehyde group, an ester, an ether, a crown ether, a ketone, an organosulfur compound, an organometallic group, a carboxylic acid, an organosilicon or a carbohydrate that optionally contains one or more alkylated hydroxyl groups; wherein said heteroalkyl, heteroaryl, and substituted heteroaryl contain at least one heteroatom that is oxygen, sulfur, a metal atom, phosphorus, silicon or nitrogen; and wherein said substituents of said substituted alkyl, substituted alkenyl, substituted alkynyl, substituted aryl, and substituted heteroaryl are halogen, CN, NO₂, lower alkyl, aryl, heteroaryl, aralkyl, aralkoxy, guanidino, alkoxycarbonyl, alkoxy, hydroxy, carboxy and amino; and wherein said amino groups are optionally substituted with an acyl group, or 1 to 3 aryl or lower alkyl groups. In certain embodiments, R₅ and R₆ are hydrogen. In other embodiments, R₁-R₁₀ are independently hydrogen, hydroxyl, methyl, ethyl, propyl, isopropyl, butyl, isobutyl, phenyl, tolyl, hydroxymethyl, hydroxypropyl, or hydroxybutyl. Non-limiting examples of the anthracenes include 1,2-dimethylanthracene, 9,10-dimethylanthracene, 7,8-dimethylanthracene, 9,10-duphenylanthracene, 9,10-dihydroxymethylanthracene, 9-hydroxymethyl-10-methylanthracene, dimethylanthracene-1,2-diol, 9-hydroxymethyl-10-methylanthracene-1,2-diol, 9-hydroxymethyl-10-methylanthracene-3,4-diol, and 9,10-di-m-tolylanthracene.

The chemical inhibitor may be introduced into the growth medium of cells. In some embodiments, the chemical inhibitor is withdrawn from the hypermutated cells or the cells are removed from the chemical inhibitor in order to restabilize the genome of the cells. Alternatively, the method may comprise inactivation of the chemical inhibitor of mismatch repair, thereby stabilizing the genome of the hypermutated cells.

MMR-suppressed cells are incubated to allow for mutagenesis. This generates hypermutated daughter cells. Preferably, MMR-suppressed cells are passaged for at least about 20 generations, more preferably at least about 30 generations, during which time genome-wide mutations accumulate in daughter cells.

The genetically diverse pool of cells are preferably subcloned by any method known in the art. For example, the hypermutated daughter cells may be single-cell subcloned by limiting dilution and expanded. Clones are preferably expanded for up to two weeks.

Daughter cells exhibiting one or more improved growth properties relative to the parental cell lines (e.g., a faster growth rate (FGR) than the parental cell population or production at high density (HD)) are then identified. In some preferred embodiments, the improved growth characteristics include both faster growth rate and production at high density. In some embodiments, the cells further demonstrate at least one characteristic of production of high affinity antibodies and high titer antibody production. For example, in some embodiments, cells exhibiting faster growing (FGR) and high antibody titer are detected. The ability to generate faster growth rate cells with high specific productivity can lead to dramatic increases in overall production for antibody manufacturing for many cell types (FIG. 2).

Preferably the identification of cells having improved growth properties comprises a high throughput screen. In some embodiments, a variety of functional, automated high throughput screening (HTS) assays are performed to identify subclones having at least one improved growth characteristic (e.g., cells yielding proteins with enhanced pharmacologic activity; cells with enhanced titer yields; faster growth rate cells; cells having enhanced growth and viability at high cell density; cells with enhanced production of biomolecules at high cell density). Such cells may be suitable for improved scaleable manufacturing.

Detection of faster growth rate daughter cells may be by any method known in the art, for example but not limited to, cell counting and comparison of populations of daughter cells relative to populations of parental cells. In some embodiments, detection of faster growth rate daughter cells having a faster growth rate than parental cells involves comparing the size of a cell population generated by a hypermutated daughter cell to the size of a cell population generated by a parental cell following an equivalent length of time in culture under like culture conditions, wherein a larger daughter cell population relative to the parental cell population is indicative of faster growth rate daughter cells. In some embodiments, detection of faster growth rate daughter cells involves identifying daughter cells having a growth ratio greater than a parental cell growth ratio. Detection of cells exhibiting enhanced production of a biomolecule at high cell density also may be by any means known in the art, for example but not limited to, comparing the cell density of a daughter cell population to the cell density of a parental cell population following an equivalent length of time in culture under like culture conditions, wherein a greater cell density of the daughter cell population is indicative of an HD cell line.

Sizes of cell populations may be determined by any means known in the art. Size of cell population is preferably determined using an optical imaging system, for example, the MetaMorph® system (Molecular Devices Corp.).

MMR is restored in subclone(s) that exhibit the desired phenotype(s). Genetic stability may be restored by removing the MMR inhibitor as previously described (8,9). For example, cells may be removed from culture medium containing a chemical inhibitor of MMR. Alternatively, the chemical inhibitor may be withdrawn from the cell. In some embodiments, a protein inhibitor of MMR is inactivated or cells are cured thereof. Genetic stability may be restored to the cells before, after, or simultaneously with detection of daughter cells exhibiting the desired phenotype.

MMR-proficient subclones exhibiting the desired phenotype are analyzed to confirm i) the preservation and stability of the cell's or antibody's enhanced properties; ii) the restoration of wild-type MMR activity and stabilization of the host's genome; and/or iii) the integrity of the protein's structure and function.

Cells produced according to the methods of the invention are included within the scope of the invention.

Methods of producing a biomolecule comprising culturing cells produced according to the methods of the invention and isolating the biomolecule from the cell or from the culture medium of the cell also are included within the scope of the invention. As used herein, the term “biomolecule” refers to a molecule produced by a cell and may comprise a chemical (e.g., a fucosyl or glycosyl moiety) and/or biological agent (e.g. a protein including but not limited to an antibody). Biomolecules produced according to the methods of the invention and pharmaceutical compositions comprising the biomolecules and pharmaceutically acceptable carriers are likewise included within the scope of the invention.

The whole genome evolution technology process allows for the development of isogenic cells that can be analyzed by a variety of genomic and proteomic tools to uncover genes and pathways that are involved in optimized cell growth or titer production. In contrast to standard chemical mutagens, which induce aneuploidy as a result of chromosomal instability (7), the whole genome evolution technology process allows comparative genetic approaches to be undertaken because it results in subtle point mutations while leaving the chromosomal stability of the host cell and long term viability intact. This feature avoids the high mutation background and mutational “hotspots” seen in chemically mutagenized cells, which result in both genetically unstable genomes as well as recurring phenotype outcomes. Not only does this approach yield more robust outcomes, it also makes differential gene/protein discovery easier by unequivocally identifying lead targets involved in pathways associated with enhanced growth and production. Included within the scope of the invention are methods of identifying genes responsible for improved growth properties of cells to which the whole genome evolution technology process has been applied. The methods comprise comparing the genome of daughter cells produced according to the methods of the invention having the desired phenotype to the genome of a parental cell and detecting genetic differences between the two genomes, wherein a gene comprising at least one of such genetic differences, or mutations, is a gene responsible for the desired phenotype. For example, whole genome evolution technology-derived mAb production cells have been used to identify pathways involved in high titer productivity by performing a RNA microarray analysis of gene expression between sets of isogenic parental and high-titer whole genome evolution technology-derived sibs (9).

The identification of evolved pathways for improved growth properties such as high titer or faster growth allows direct engineering of high-performance cells for high production of many products. This can be achieved by discovering modified pathways in whole genome evolution technology-derived cells with enhanced properties and recapitulating the mutant phenotype by cell engineering these pathways onto a parental cell backbone. For example, the genes responsible for improved growth characteristics identified by the methods described herein can be recombinantly expressed in cells, preferably production cells, by methods known in the art. Recombinant expression of the genes responsible for improved growth characteristics in parental cell lines (either via whole genome evolution technology or directed pathway modifications resulting from whole genome evolution technology) can in turn accelerate the speed to the clinic by reducing the time required to generate stable production cell lines.

The following example describes several aspects of embodiments of the invention in greater detail. The example is provided to further illustrate, not to limit, aspects of the invention described herein.

### EXAMPLE

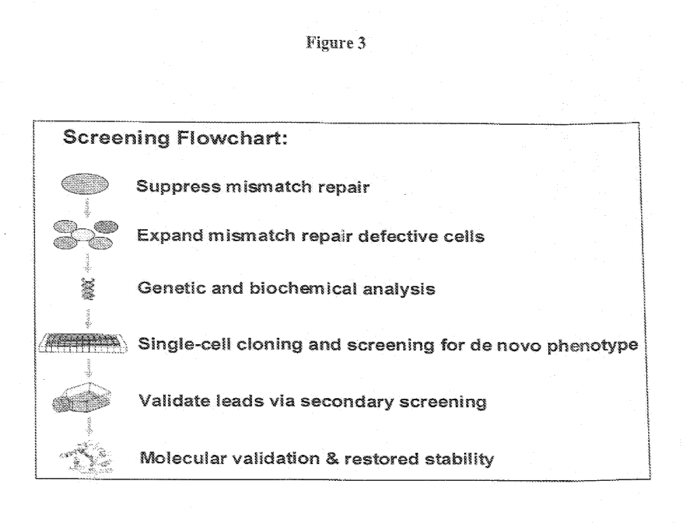

The whole genome evolution technology process was applied as outlined in FIG. 3. MMR was suppressed in parental cells using the MMR-chemical inhibitor 9, 10-dimethylanthracene. The MMR-suppressed cell line was propagated and subsequently subcloned to yield approximately 10,000 sibling cells that were screened for a Faster growth rate phenotype using a customized visualization platform and software that can monitor cell growth at low density.

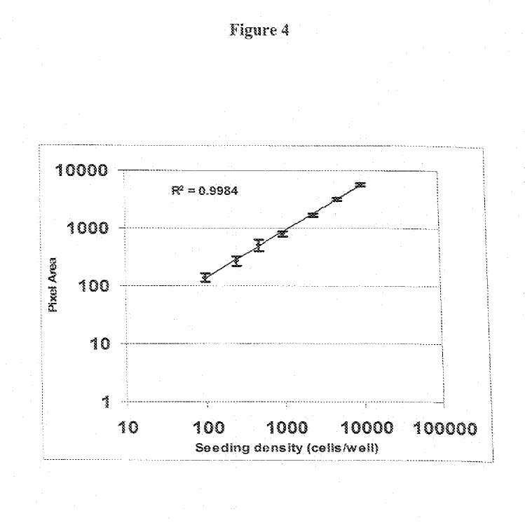

To screen for the faster growth rate (FGR) clones in a whole genome evolution technology-derived CHO-MAb cell pool, a new image-based HTS method has been developed. This method avoids the requirement of traditional time-consuming cell counting methods. Instead, it uses the Meta Imaging System (Molecular Devices, Downingtown, Pa.) which images cell colony size via a digital camera that is interfaced to an ORCA automated platform (Beckman Coulter, Fullerton, Calif.) designed to plate, feed, and analyze colony sizes of sib cell clones generated via whole genome evolution technology under sterile conditions. Using the Metamorph software package (Ver.6.3r0) the colony area image is quantified using pixels and exported to spreadsheet that calculates ratios of cell colony size during a 3 day growth period, comparing size of day 14 and day 17. The imaging system has been refined to generate a linear correlation between the image pixel area and cell number (FIG. 4).

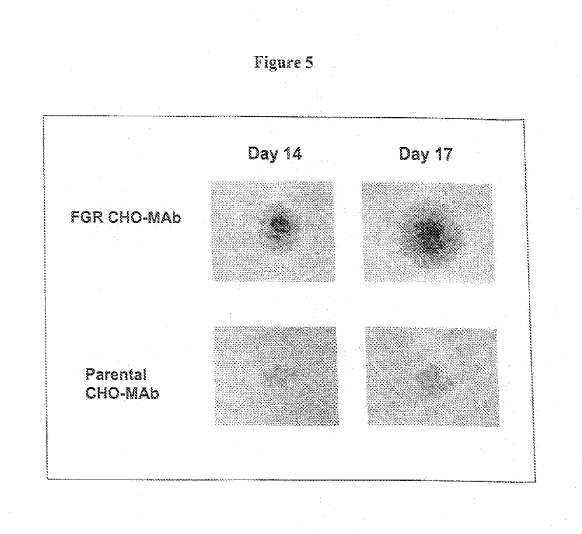

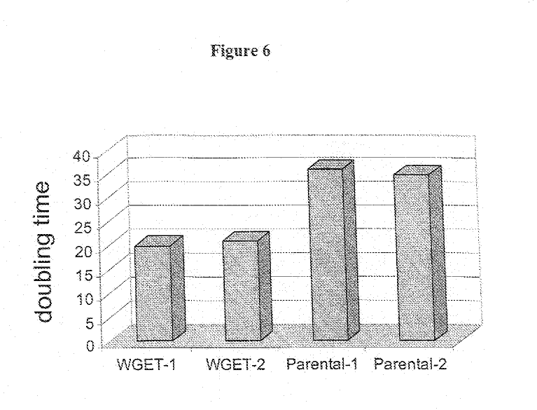

To identify Faster growth rate sibs in whole genome evolution technology-derived CHO-MAb cells, cells are seeded into 100 to 200 bar-coded round-bottom 96-well plates at 0.8 cells/well using the Biomek FX robotic system (Beckman Coulter, Fullerton, Calif.) to ensure for single cell clones per well. The imaging method does not require sacrificing the cells for quantification, therefore, replica plates are not required using this procedure. Two weeks after initial clonal seeding, plates are screened by the ORCA system for high throughput analysis of clone growth as determined by microscopic imaging. Plates are housed in stacking incubators and returned to the incubator after image analysis without any disturbance of the colonies. This step is repeated three days later to determine the size of expansion of colonies which is then used to determine cell growth ratios between day 14 and 17 time points. Cell growth is calculated as the ratio between the colony size value measured at day 17 and day 14, by dividing the colony area at day 17 by the colony area at day 14 by 3 days. FIG. 5 illustrates a typical analysis where parental or FGR CHO-MAb clones are imaged at day 14 and then again at day 17. As expected, the FGR cells show a larger ratio between day 17 and 14 as compared to clones derived from parental CHO-MAb. On average for this cell line, parental clones show a growth ratio of ˜0.5 as determined through a primary screening of approximately 20,000 independent parental CHO-MAb-derived subclones, whereas FGR clones exhibit a growth ratio of >0.8. As a standard, each plate contains clones derived from parental CHO-MAb cells as comparator. FIG. 5 shows a typical profile of subclones derived from the parental line and whole genome evolution technology-derived cells. In whole genome evolution technology-derived CHO-MAb cells, ˜5% of the wells screened exhibit a growth ratio of 0.8 or higher. Of these leads, 50% were confirmed to have improved growth rate compared with that of the parental line when expanded which is consistent with screens for other cell lines using this assay. Confirmed clones are grown in 3 mL static cultures during a 48 hour-quantitative proliferation assay whereby cells are physically counted at day 0, 1 and 2 using Cedex automated cell counter. FIG. 5 shows a representative 48 hour-quantitative proliferation assay result. The top performing Faster growth rate clones are further expanded and evaluated in a 20 mL shake flask assay. The majority of clones that reach this level typically maintain their faster growth rate while retaining its high antibody specific productivity (FIG. 6).

Structural analysis of antibodies derived from Faster growth rate subclones confirms that antibodies produced by these cells retain similar genetic and biochemical properties to that of the parental antibody. In addition, extended culturing of Faster growth rate cells demonstrate that the enhanced growth rate mediated by the whole genome evolution technology was stable and that the overall titers of cells during a 3 month growth period remained constant in both parental CHO and Faster growth rate derived cells (FIG. 6).

### REFERENCES

1. Maynard J, and Georgiou G. Antibody engineering. Ann. Rev. Biomed. Eng. 2000. 2:339-76.
2. Ma, J K, Drake, P M, Christou, P. The production of recombinant pharmaceutical proteins in plants. Nat. Rev. Genet. 2003. 4:794-805.
3. Evans, D. & Das, R. Monoclonal Antibodies: Evolving into a $30 Billion Market (DataMonitor, London, U.K., 2005).
4. Davies, J. & and Reff, M. Chromosome localization and gene-copy-number quantification of three random integrations in Chinese hamster ovary cells and their amplified cell lines using fluorescence in situ hybridization. Biotechnol. Appl. Biochem. 2001. 33, 99-105
5. Lonberg, N. Human antibodies from transgenic animals. Nat. Biotech. 2005. 23:1117-1125.
6. Zhu, L. et. al. Production of human monoclonal antibody in eggs of chimeric chickens. Nat. Biotech. 2005. 23:1159-1169.
7. Bardelli A, Cahill D P, Lederer G, Speicher M R, Kinzler K W, Vogelstein B, Lengauer C. Carcinogen-specific induction of genetic instability. Proc. Natl. Acad. Sci. 2001. 98:5770-5.
8. Nicolaides, N. C., Ebel, W., Kline, J. B., Chao, Q., Routheir, E., Sass, P. M., and Grasso, L. Morphogenics as a Tool for Drug Discovery. Ann. N.Y. Acad. Sci. 2005. 1059:1-11.
9. Grasso, L., Kline, J. B., Chao, Q., Routheir, E., Ebel, W., Sass, P. M., and Nicolaides, N. C. MORPHODOMA Technology: Enhancing Therapeutic Antibodies and Titer Yields of Mammalian Cell Lines. Bioprocessing Intl. 2004. 2:58-64.
10. Li, J., Sai, T., Berger, M., Chao, Q., et. al. Human Antibodies for Immunotherapy Development Generated via a Human B-Cell Hybridoma Technology. Proc. Natl. Acad Sci USA. 2006. (in press).
11. Nicolaides, N. C., Carter, K., Shell, B. K., Papadopoulos, N., Vogelstein, B., and Kinzler, K. W. Genomic Organization of the Human PMS2 Gene Family. Genomics. 1995. 30:195-206.
12. Nicolaides N C, Littman S J, Modrich P, Kinzler K W, Vogelstein B. A naturally occurring hPMS2 mutation can confer a dominant negative mutator phenotype. Mol. Cell. Biol. 1998. 18:1635-41.
13. Chao, Q., Sullivan, C., Getz, J., Gleason, K., Sass, P. M., Nicolaides, N. C., and Grasso, L. Rapid generation of Plant Traits via Regulation of DNA Mismatch Repair. Plant Biotechnology. 2005. 3:399-407.
14. Wurm, F. M. Production of recombinant protein therapeutics in cultivated mammalian cells. Nature Biotechnology. 2004. 22:1393-1398.
15. U.S. Pat. No. 6,808,894.
16. U.S. patent application Ser. No. 10/714,228.
17. U.S. patent application Ser. No. 10/901,650.
18. Kohler et al., Nature, 256: 495, 1975.
19. Marks et al., J. Mol. Biol., 222: 581-597, 1991.
20. Garmestani et al., Nucl. Med. Biol., 28: 409, 2001.
21. Kreitman, R. J. Adv. Drug Del. Rev., 31: 53, 1998.
22. Morrison et al., Proc. Natl. Acad. Sci. U.S.A., 81: 6851-6855, 1984.
23. Jones et al., Nature, 321: 522-525, 1986.
24. Reichmann et al., Nature, 332: 323-329, 1988.
25. Presta, Curr. Op. Struct. Biol., 2: 593-596, 1992.
26. Muller (1980) J. Immunol. Meth. 34:345-352.
27. Antoni and Mariani (1985) J. Immunol. Meth. 83:61-68.
28. Urlaub and Chasin (1980) Proc. Natl. Acad. Sci. USA, 77: 4216.
29. Graham et al., (1977) J. Gen Virol., 36: 59.
30. Mather, (1980) Biol. Reprod., 23:243-251.
31. Mather et al. (1982) Annals N.Y. Acad. Sci. 383:44-68.
32. Nicolaides et al. (1995) Genomics 30:195-206.
33. Horii et al. (1994) Biochem. Biophys. Res. Commun. 204:1257-1264.

What is claimed:

1. A method for generating cells having an improved growth property relative to a parental cell population comprising:
a. inhibiting mismatch repair of a parental cell of said parental cell population;
b. incubating said parental cell to allow for mutagenesis, thereby generating hypermutated daughter cells;
c. detecting hypermutated daughter cells having said improved growth property; and
d. restoring genetic stability of said hypermutated daughter cells having said improved growth property.

2. The method of claim 1 wherein said improved growth property is faster growth rate.

3. The method of claim 1 wherein said improved growth property is enhanced production of a biomolecule at high cell density.

4. The method of claim 1 wherein said improved growth property is enhanced cell viability at high cell density.

5. The method of claim 1 wherein said parental cell is an antibody-producing cell.

6. The method of claim 1 wherein said step of inhibiting mismatch repair of said parent cell comprises exposing said parental cell to a chemical inhibitor of mismatch repair.

7. The method of claim 1 wherein said step of inhibiting mismatch repair of said parent cell comprises exposing said parental cell to a protein inhibitor of mismatch repair.

8. The method of claim 1 wherein said step of incubating comprises passaging said hypermutated cells for at least 20 passages.

9. The method of claim 6 wherein said step of restoring genetic stability comprises withdrawing said chemical inhibitor from said hypermutated daughter cells.

10. The method of claim 7 wherein said step of restoring genetic stability comprises inactivating said protein inhibitor.

11. The method of claim 1 wherein said step of restoring genetic stability occurs before said step of detecting hypermutated daughter cells having the improved growth property.

12. The method of claim 1 wherein said step of restoring genetic stability occurs after said step of detecting hypermutated daughter cells having the improved growth property.

13. The method of claim 1 wherein said step of detecting hypermutated daughter cells having the improved growth property comprises a high throughput screen for said hypermutated daughter cells having the improved growth property.

14. The method of claim 2 wherein said step of detecting hypermutated daughter cells having a faster growth rate comprises comparing the size of a cell population generated by a hypermutated daughter cell to the size of a cell population generated by a parental cell following an equivalent length of time in culture under the same culture conditions, wherein a more dense daughter cell population is indicative of said faster growth rate.

15. The method of claim 2 wherein said step of detecting hypermutated daughter cells having a faster growth rate comprises identifying said daughter cells having a growth ratio greater than a parental cell growth ratio.

16. The method of claim 14 wherein the size of said daughter cell population is determined using an optical imaging system.

17. The method of claim 14 wherein the size of said parental cell population is determined using an optical imaging system.

18. The method of claim 1 wherein said parental cell is a mammalian cell.

19. The method of claim 1 wherein said parental cell is a hybridoma cell.

20. A cell produced according to the method of claim 1.

21. A method of manufacturing a biomolecule comprising culturing the cell of claim 20 and isolating said biomolecule from said cell or culture medium of said cell.

22. The method of claim 21 wherein said biomolecule comprises a chemical agent.

23. The method of claim 22 wherein said biomolecule comprises a biological agent.

24. The method of claim 21 wherein said biomolecule comprises a biological agent.

25. The method of claim 24 wherein said biological agent comprises a protein.

26. The method of claim 24 wherein said biological agent comprises an antibody.

27. A biomolecule produced according to the method of claim 21.

28. A pharmaceutical composition comprising the biomolecule of claim 27 and a pharmaceutically acceptable carrier.

29. A method of identifying genes responsible for an improved growth property comprising comparing the genome of the cell of claim 20 to the genome of said parent cell to identify mutations, wherein a gene responsible for the improved growth property comprises at least one of said mutations.
